(19) United States
(12) Patent Application Publication
(10) Pub. No.: US 2022/0323312 A1
(43) Pub. Date: Oct. 13, 2022

(54) SUN CARE COMPOSITIONS WITH UV REFLECTIVE PARTICLES

(71) Applicants: DOW GLOBAL TECHNOLOGIES LLC, Midland, MI (US); ROHM AND HAAS COMPANY, Collegeville, PA (US)

(72) Inventors: Yuanqiao Rao, Collegeville, PA (US); Nikhil J. Fernandes, Collegeville, PA (US); Hyunwoo Kim, Freeport, TX (US); Lei Liu, Freeport, TX (US)

(73) Assignees: DOW GLOBAL TECHNOLOGIES LLC, Midland, MI (US); ROHM AND HAAS COMPANY, Collegeville, PA (US)

(21) Appl. No.: 17/621,035

(22) PCT Filed: Jun. 25, 2020
(86) PCT No.: PCT/US2020/039627

§ 371 Date: Dec. 20, 2021

Related U.S. Application Data
(60) Provisional application No. 62/866,267, filed on Jun. 25, 2019.

(51) Int. Cl. A61K 8/02; A61Q 17/04; A61K 8/81

(52) U.S. Cl. CPC A61K 8/0241; A61Q 17/04; A61K 8/8152; A61K 2800/41

(57) ABSTRACT

Described herein are sun care compositions, comprising UV reflective particles ground from an extruded film having a plurality of alternating layers of polycarbonate (PC) and poly(methyl methacrylate) (PMMA), wherein each layer is less than 150 nm thick; and at least one of a cosmetically acceptable emollient, humectant, vitamin, moisturizer, conditioner, oil, silicone, suspending agent, surfactant, emulsifier, preservative, rheology modifier, pH adjustor, reducing agent, anti-oxidant, and/or foaming or de-foaming agent, and methods of making and using the sun care compositions.

### CROSS-REFERENCE TO RELATED APPLICATIONS

This application claims priority to U.S. Provisional Application Ser. No. 62/866,267, filed Jun. 25, 2019, the entire contents of which are incorporated by reference herein.

### BACKGROUND

Sun care compositions are typically personal care compositions designed to prevent a percentage of ultraviolet (UV) radiation coming from the sun from reaching the wearer's skin. UVA radiation (315 nm-400 nm) does not cause visible radiation burns (e.g., sunburn), but has been shown to cause indirect DNA damage through free radical generation. UVB radiation (290 nm-315 nm) causes sunburn in the short term, and is additionally associated with cancers (e.g., melanomas) over time. Sunscreen sun protection factor (SPF) ratings are relevant to UVB blocking.

However, with growing awareness of the dangers of UVA damage, it is important to develop sun care actives (sometimes referred to as sunscreen agents) that can effectively address both UVB and UVA radiation.

### SUMMARY

Described herein are sun care compositions comprising UV reflective particles ground from an extruded film having a plurality of alternating layers of polycarbonate (PC) and poly(methyl methacrylate) (PMMA), wherein each layer is less than 150 nm thick; and at least one of a cosmetically acceptable emollient, humectant, vitamin, moisturizer, conditioner, oil, silicone, suspending agent, surfactant, emulsifier, preservative, rheology modifier, pH adjustor, reducing agent, anti-oxidant, and/or foaming or de-foaming agent, and methods of making and using the sun care compositions.

### BRIEF DESCRIPTION OF THE DRAWINGS

FIG. 1 is an Atomic Force Microscope (AFM) image of a cross section of a UV reflective film.

FIG. 2 is a UV-Vis reflection spectrum of the UV reflective film.

FIG. 3 is a UV-Vis transmission spectrum of the UV reflective film.

FIG. 4 is a UV-Vis transmission spectrum of the UV reflective film and a comparative polycarbonate film.

FIG. 5 is a UV-Vis reflection spectrum of another UV reflective film.

FIG. 6 is an optical micrograph of UV reflective particles produced from the UV reflective film of FIG. 5.

FIG. 7 is a UV-Vis transmission spectrum of a sun care composition incorporating UV reflective particles produced from the UV reflective film of FIG. 5 and a control personal care composition.

### DETAILED DESCRIPTION

Described herein are sun care compositions comprising UV reflective particles according to the present disclosure. It is understood that UV reflective can be used synonymously with UV blocking, as the important feature is curtailing UV transmission through the composition containing the particles, as will be described.

A sun care composition is a personal care composition for protecting a user from UV radiation. Examples of sun care compositions include compositions having an SPF rating (for example, sunscreen compositions) and/or personal care compositions where a UV blocker would he beneficial, such as, for example, moisturizers, lip balms, etc.

A sun care composition rosy contain at least one of a cosmetically acceptable emollient, humectant, vitamin, moisturizer, conditioner, oil, silicone, suspending agent, opacifier/pearlizer, surfactant, emulsifier, preservative, rheology modifier, colorant, pH adjustor, propellant, reducing agent, anti-oxidant, fragrance, foaming or de-foaming agent, tanning agent, insect repellant, and/or biocide. In an embodiment, a sun care composition may contain at least one of a humectant, a surfactant, and/or an emollient.

In an embodiment, the sun care composition contains one or more additional sunscreen actives. Suitable sunscreen actives include titanium dioxide, zinc oxide, oxybenzone, dioxybenzone, cinoxate (2-ethoxyethyl-p-methoxy-cinnamate), diethanolamine-p-methoxycinnamate, ethylhexyl-p-methoxy-cinnamate, isopentenyl-4-methoxycinnamate, 2-ethylhexyl salicylate, digalloyl trioleate ethyl 4-bis(hydroxypropyl)aminobenzoate, glyceryl aminobenzoate, methyl anthranilate, homosalate (3,3,5-trimethylcyclohexyl salicylate), triethanolamine salicylate, 2-phenyl-benzimidazole-5-sulfonic acid, sulisobenzone (2-hydroxy-4-methoxy-benzophenone-5-sulfonic acid), Padimate A (amyl p-dimethylaminobenzoate), Padimate O (octyl dimethyl pars aminobenzoate), Octocrylene (2-ethylhexyl-2-cyano-3,3 diphenylacrylate), Avobenzone, (4-t-butyl-4′-methoxydibenzoyl-methane), Ecamsule, 4-Methylbenzylidene camphor, Tinosorb M, Tinosorb S, Neo Heliopan AP, Mexoryl XL, Benzophenone-9, Uvinul T 150, Uvinul A Plus, Uvasorb HEB, and/or Parsol SLX, and mixtures thereof.

UV reflective particles of the present disclosure are ground particles of a UV reflective film having a series of alternating layers of two polymers with different refractive indexes. The number of two-polymer pair layers (e.g., a layer of a second polymer over a layer of a first polymer) may be greater than about 50, greater than about 100, greater than about 150, greater than about 200, greater than about 300, greater than about 400, and less than about 450, less than about 350, less than about 250, and less than about 220, embracing all combinations and sub-combinations therein. In an embodiment, the number of pair layers may be about 200.

Each individual polymer layer may have a thickness from about 35 nm to about 160 nm, preferably from about 40 nm to about 100 nm, more preferably from about 50 nm to about 90 nm. In an embodiment, each individual polymer layer is less than 150 nm thick. In an embodiment, the layers are fairly uniform in thickness, for example, the variation in thickness of each of the layers (e.g., for a given polymer) may be less than about 50%, preferably less than about 35%. It is understood that consistency in layer thickness throughout the film is desirable. Extrusion process conditions, such as, for example, die gap, flow rate, and temperature, affect the thickness of individual polymer layer.

In an embodiment, one of the polymers is polycarbonate (PC). Commercially available polycarbonate resins include, for example, CALIBRE™ 200-14 polycarbonate resin (commercially available from Trinseo LLC, Berwyn, Pa.), a natural polycarbonate, resin with a melt flow index (MFI) of 14 dg/min (300° C./1.2 kg), and CALIBRE™ 302-10 polycarbonate resin (commercially available from Trinseo LLC, Berwyn, Pa.), a polycarbonate resin with an MFI of 10 dg/min (300° C./1.2 kg).

In an embodiment, one of the polymers is an (meth)acrylic polymer. In an embodiment, one of the polymers is poly(methyl methacrylate) (PMMA). Commercially available PMMA resins include, for example, PLEXIGLAS™ V045-100 acrylic resin (commercially available from Arkema, Inc., King of Prussia, Pa.), a poly(methyl methacrylate) resin with a MFI of 2.3 dg/min (230° C./3.8 kg), and PLEXIGLAS™ V825 acrylic resin (commercially available from Arkema, Inc., King of Prussia, Pa.), a poly(methyl methacrylate) resin with a MFI of 3.7 dg/min (230° C./3.8 kg).

In an embodiment, the alternating layers form a core structure of alternating layers of different refractive indexes (e.g., a core structure of alternating layers of polycarbonate and PMMA). The core structure may be covered with at least one skin layer. The core structure may be sandwiched between two skin layers. In an embodiment, the skin layer may comprise one or more of the polymers selected from those comprising the alternating layers. In an embodiment, the skin layer may comprise an (meth)acrylic polymer. In an embodiment, the skin layer may comprise PMMA. In an embodiment, the skin layer may comprise a polymer that is neither of the polymers comprising the alternating layers (e.g., a third polymer). In an embodiment, the skin layer may comprise a polymer with a different refractive index from either of the polymers comprising the alternating layers.

The skin layer(s) may be thicker than a two-polymer pair layer of alternating polymers, in an embodiment, the thickness of the skin layers (e.g., both together) compared to the thickness of the total film may be greater than about 5%, greater than about 7%, greater than about 10%, and less than about 50%, less than about 20%, and less than about 12%, embracing all combinations and sub-combinations therein. For example, if the total film (e.g., core structure plus skin layers) thickness is less than 100 microns, in an embodiment, the skin layers may be less than about 10 microns.

To create the film, the alternating layers may be formed by coextruding a PC resin feed from a first extruder and a PMMA resin feed from a second extruder through a feedblock and/or a multiplier. The use of a multiplier allows the desired number of layers to be generated. In an embodiment, the coextrusion via feedblock and multiplier is used to form at least 50 alternating liquid resin pair layers (e.g., a total of 100 individual polymer layers). The number of two-polymer pair layers may be greater than about 50, greater than about 100, greater than about 150, greater than about 200, greater than about 300, greater than about 400, and less than about 450, less than about 350, less than about 250, and less than about 220, embracing all combinations and sub-combinations therein. In an embodiment, the coextrusion via feedblock and multiplier is used to form about 102 alternating liquid resin pair layers (e.g., a total of about 205 individual polymer layers). The alternating layers may be cast on a chill roll.

The term “median particle size” as used herein and in the appended claims is the D50 value determined in accordance with ASTM B822-17.

The UV reflective particles may be formed by forming a UV reflective film as described herein and then grinding the film. For example, a film may be cut into pieces (for example, having a size less than 5 mm). A high-speed dry rotor-stator mill may be used to grind the film pieces into particles. Preferably, the film pieces are ground until the resulting particles pass through a sieve with trapezoidal holes of 0.5 mm (preferably, 0.25 mm; more preferably, 0.20 mm; still more preferably, 0.12 mm; most preferably, 0.08 mm). In an embodiment, the film is ground into particles having a median particle size less than 200 microns. In an embodiment, the film is ground until the particles have a median particle size of about 120 microns. It is understood that grinding can be used to achieve a variety of desired median particle sizes that still fall within this description.

In an embodiment, the UV reflective particles are ground until the median particle size is less than 100 microns. An aspect ratio of a particle refers to its shortest three-dimensional axis versus its longest three-dimensional axis (for example, thickness versus length). In this definition of aspect ratio, the orientation of layers within the particle (e.g., grain) are irrelevant to determining which axis is the shortest and longest. Accordingly, aspect ratios of the UV reflective particles may vary from an aspect ratio of 1 to about 1 (e.g., when finely ground) to an aspect ratio of 1 to about 100. In an embodiment, the UV reflective particles have an aspect ratio of about 1 to about 10, about 1 to about 8, about 1 to about 5, about 1 to about 4, about 1 to about 3, about 1 to about 2, and/or about 1 to about 1.5. In an embodiment, the UV reflective particle shape has an aspect ratio that is greater than an aspect ratio of about 1 to about 2. In an embodiment, the UV reflective particle shape has an aspect ratio of about 1 to about 5. Without being bound by theory, it is believed that a relatively larger aspect ratio is desirable for UV blocking,

In an embodiment, the present sun care compositions contain greater than about 5%, greater than about 7%, greater than about 9%, and less than about 20%, less than about 15%, and less than about 12%, UV reflective particles by weight of the composition. In an embodiment, the present sun care compositions contain about 10% UV reflective particles by weight of the composition. In an embodiment, the UV reflective particles block a portion of at least one of UVA radiation and/or UVB radiation. In an embodiment, the sun care composition is highly transparent in the visible light range, for example to avoid imparting a whitening effect on a user's skin (e.g., which may be aesthetically undesirable).

In use, sun care compositions including UV reflective particles described herein may be used to protect a mammal from damage caused by UV radiation (e.g., UVA radiation and/or UVB radiation). For example, a method of protecting a mammal (e.g., the skin of a mammal) from damage caused by UV radiation comprises applying the presently described sun care compositions to the skin of the mammal.

The following examples are for illustrative purposes only and are not intended to limit the scope of the appended claims.

### EXAMPLES

### Example 1

A first 31.75 mm (1.25 inch) diameter, 24:1 L/D single screw extruder was loaded with a polycarbonate resin. The polycarbonate resin was dried in a drier at 120° C. overnight to reduce the moisture content before processing.

A second 31.75 mm (1.25 inch) diameter, 24:1 L/D single screw extruder was loaded with a poly(methyl methacrylate) (PMMA) resin. The PMMA resin was dried in a drier at 80° C. overnight to reduce the moisture content before processing.

The first extruder and second extruder were arranged to form a coextrusion line used for a microlayer extrusion. A sequential feedblock and layer multipliers were used to produce layered coextruded structures with about 102 alternating liquid resin pair layers (e.g., a total of about 205 individual polymer layers).

The coextruded structures were merged with skin layers comprising PMMA resin extruded at 20 lb/h from an 8-inch wide film die to create 12-50-micron thick films, Extruder and die temperatures were set at 243° C. The extruders fed individual gear pumps to ensure uniform flow of the polymer melts to the feedblock and dies. Extruded films were cooled down on a chill roll set at 104° C. The resulting UV reflective films had alternating layers of polycarbonate and poly(methyl methacrylate).

Film 1 comprised a core structure of CALIBRE™ 200-14 polycarbonate resin and PLEXIGLAS™ V045-100 acrylic resin (e.g., PMMA) alternating layers, with PMMA skin layers (also of PLEXIGLAS™ V045-100 acrylic resin) on either side of the core structure. The extrusion process conditions, such as, for example, die gap, flow rate, and temperature, were controlled to generate a film where the thickness of individual polymer layer was about 60 nm.

Film 2 comprised a core structure of CALIBRE™ 200-14 polycarbonate resin and PLEXIGLAS™ V825-100 acrylic resin (e.g., PMMA) alternating layers, with PMMA skin layers (also of PLEXIGLAS™ V825-100 acrylic resin) on either side of the core structure. The extrusion process conditions, such as, for example, die gap, flow rate, and temperature, were controlled to generate a film where the thickness of individual polymer layer was about 67 nm.

### Example 2 (Comparative)

A comparative Film A was prepared. A 31.73 mm (1.25 inch) diameter, 24:1 L/D single screw extruder was loaded with CALIBRE™ 200-14 polycarbonate resin and was extruded as a comparative, single refractive index film. The resulting film was a single-polymer (e.g., polycarbonate) film with a thickness of about 25 micron. Comparative Film A did not have multiple layers.

### Example 3

Film 1 (described in Example 1) was prepared substantially according to Example 1 and characterized as follows using an Atomic Force Microscope (AFM). Cross-sectioned multilayer film samples were prepared by punching specimens out of samples and mounting them in vise holders. Samples were milled flat with a cryo-mill at ˜−80° C. The samples were then polished with cryomicrotomy at −80° C. The block faces were examined. Peak force tapping AFM images were obtained on a BRUKER ICON™ atomic force microscope using a NANOSCOPE V™ controller (software v 8.15). NANOWORLD ARROW NCR™ cantilevers were used with the following settings: PF setpoint of 0.045, scan assist auto gain on, PF amplitude of 300, DMT mod limit of 4, Z range of 4, and PF engagement setpoint of 0.1. All images were captured at 1024 lines of resolution and produced with SPIP version 6.4.2. software. A second order average plane fit was used, with a zero order LMS and the, mean set to zero. Layer measurements were performed using an IMAGEJ™ Java image processing and analysis program.

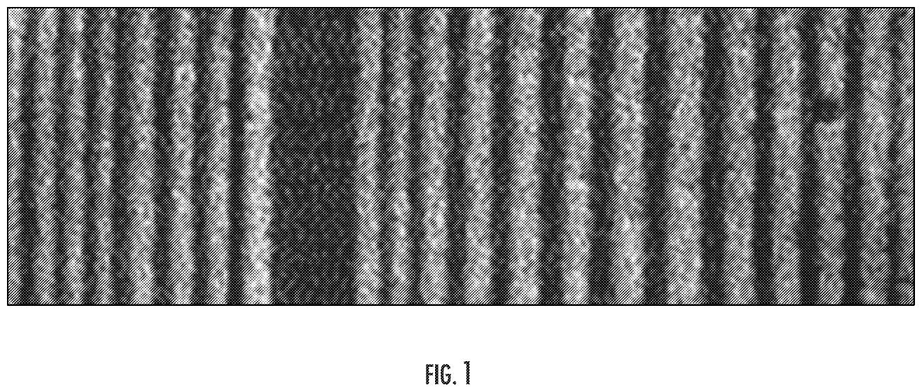

The total thickness of Film 1 was determined to be 25 microns. Film 1 contained a 102-layer pair core that was about 12 microns thick. The layers varied in individual thickness, the variation being is 5914+/−18 nm. This demonstrated satisfactory control of the layer thickness, as the nominal thickness of the individual layers was designed to be 60 nm. FIG. 1 is an AFM image of a cross section of Film 1. As can be seen in FIG. 1, Film 1 has a series of alternating layers of different refractive indexes (e.g., alternating layers of polycarbonate and PMMA).

### Example 4

UV-Vis spectroscopy was undertaken on a Film 1 (described in Example 1) prepared substantially according to Example 1. Diffuse transmission spectra were acquired with a PERKINELMER LAMBDA 950™ UV-Vis-NIR spectrometer and a 60 mm integrating sphere accessory at a resolution (slit width) of 2 nm and data interval of 2 nm. The detector response time was 0.2 sec. Approximately eighty reflection and transmission data were collected.

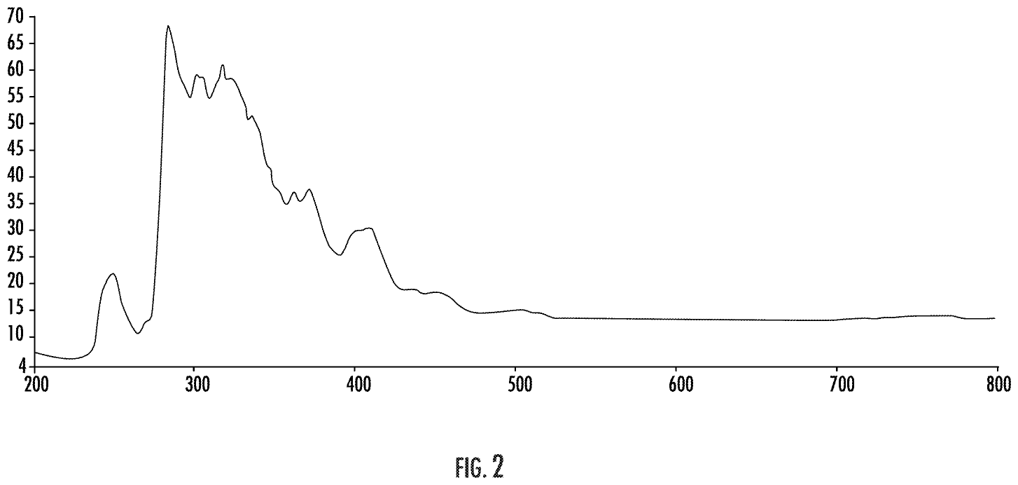

FIG. 2 is a UV-Vis reflection spectrum of the UV reflective film. FIG. 2 shows that Film 1 has strong UV reflection in the range of 290 to 400 nm, while very low reflection in the visible range (<15% reflection between 450 nm and 800 nm). Accordingly, Film 1 is highly transparent in the visible range (˜90% transmission at 550 nm), which is desirable aesthetically:

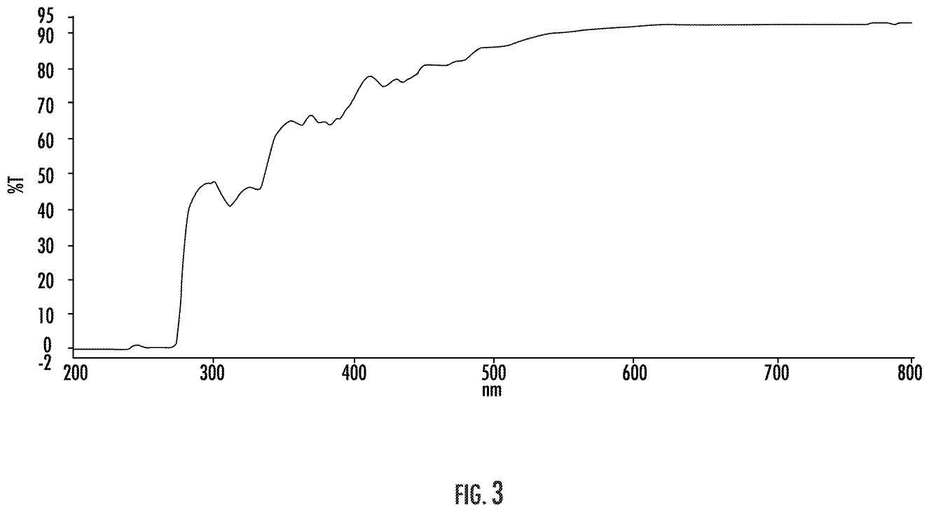

FIG. 3 is a UV-Vis transmission spectrum of the UV reflective film. Transmission between approximately 290 nm and 400 nm qualify the performance of UV blocking. As shown in FIG. 3, Film 1 exhibits a strong UV blocking effect.

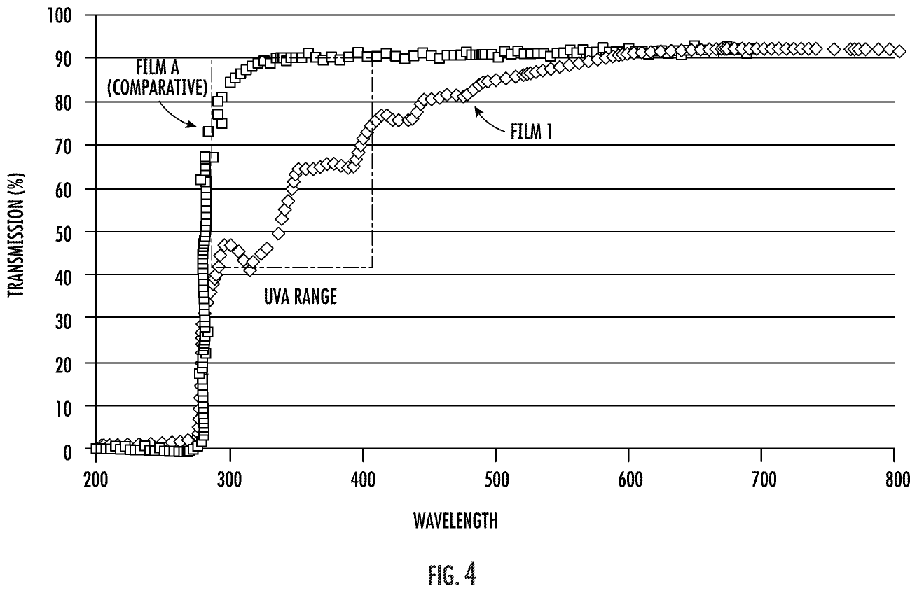

A comparative Film A was prepared substantially according to Example 2 and subjected to UV-Vis spectroscopy. FIG. 4 is a UV-Vis transmission spectrum of Film 1 and comparative Film A. As shown in FIG. 4, comparative Film A does not block transmission in the UV spectrum (e.g., the transmission is above 85% in the UV range (290 nm to 400 nm)). In contrast, Film 1 exhibits a strong UV blocking effect.

### Example 5

Film 2 (described in Example 1) was prepared substantially according to Example 1, except that nominal thickness of each individual layer was designed to be 67 nm with a 104-layer pair core. The thickness of the Film 2 was about 28 micron. Film 2 was characterized as follows using a UV-Vis reflection spectrum. Diffuse transmission spectra were acquired with a PERKINELMER LAMBDA 950™ UV-Vis-NIR spectrometer and a 60 mm integrating sphere accessory at a resolution (slit width) of 2 nm and data interval of 2 nm. The detector response time was 0.2 sec. Approximately 80 reflection data were collected.

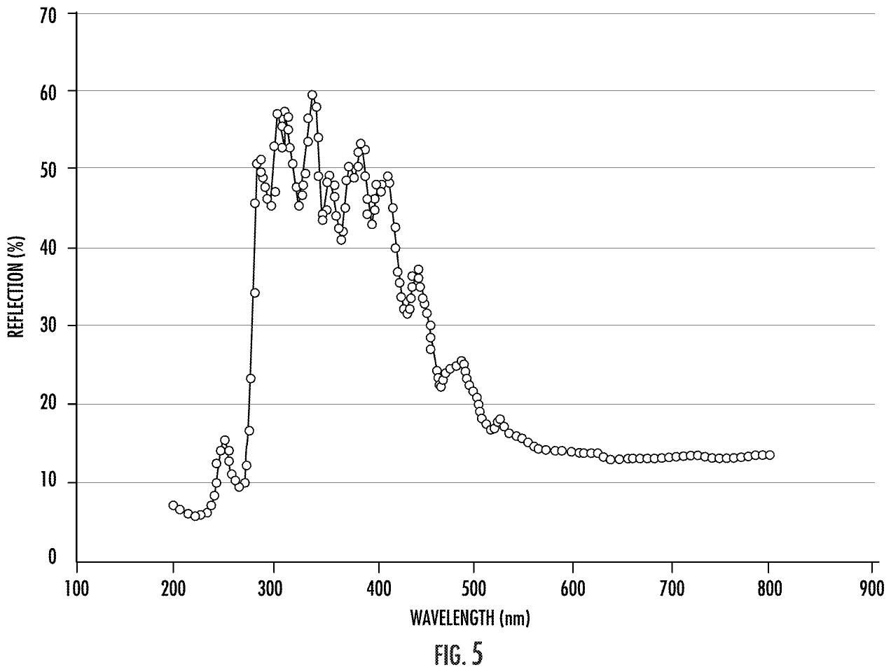

FIG. 5 is a UV-Vis reflection spectrum of Film 2. As shown, Film 2 exhibits a broad and stable UV reflection in the range of 290 to 400 nm (an average of 50%). The breadth of the UV reflection is advantageous in blocking broad solar UV radiation.

To fabricate UV reflective particles, Film 2 was cut into pieces of a size less than 5 mm. A RETSCH ZM 200™ high-speed dry rotor-stator mill was used to further reduce the pieces into UV interference particles. Both dry ice and liquid nitrogen were used as the grinding media. The grinding speed was set between 6000 rpm-18000 rpm and the screen sizes with trapezoidal holes of 2000, 1000, 500, 120 and 80 microns were used, resulting in a film powder comprising UV reflective particles.

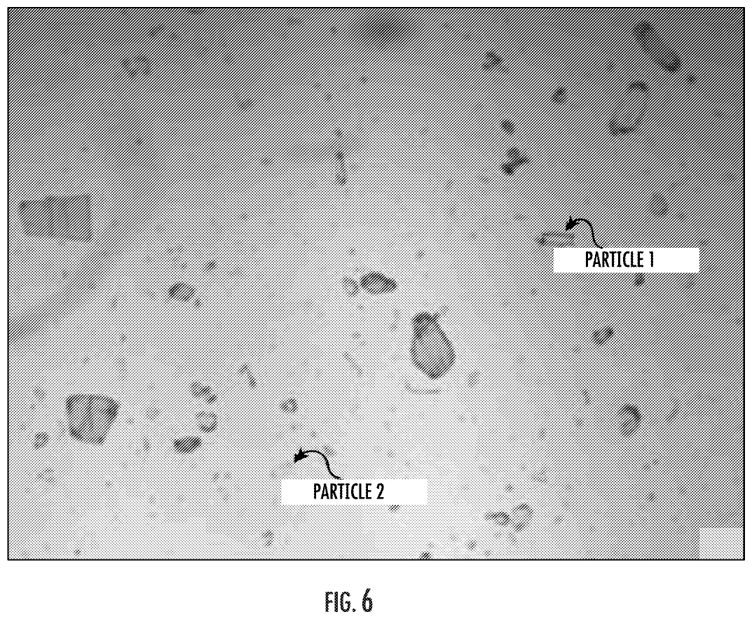

After forming the UV reflective particles, the particles were added in deionized (DI) water. A drop of MICRO90™ cleaning solution containing a surfactant was added to the particle and water mixture to wet the film powder. The mixture was then sonicated for 5 min, then it was stirred on a stir plate. While stirring, the mixture was pipetted into a BECKMAN COULTER LS 13 320™ laser diffraction particle size analyzer to determine particle size measurement, Fraunhofer diffraction was used to calculate the particle size. A median particle size (D22) of 122 microns was measured using the particle sizer. Smaller particles could be produced similarly if desired. FIG. 6 is an optical micrograph of the particles produced from Film 2. FIG. 6 illustrates successful formation of small individual particles. Particle 1 was about 170 microns. Particle 2 was about 71 microns.

### Example 6

To ascertain the UV blocking efficiency of UV reflective particles, UV reflective particles were formed substantially as described in Example 5.

These UV reflective particles were added a NIVEA™ daily moisture body lotion via speed mixer at a loading of 10% to create a sun care composition.

Untreated NIVEA™ daily moisture body lotion was used as a control sample.

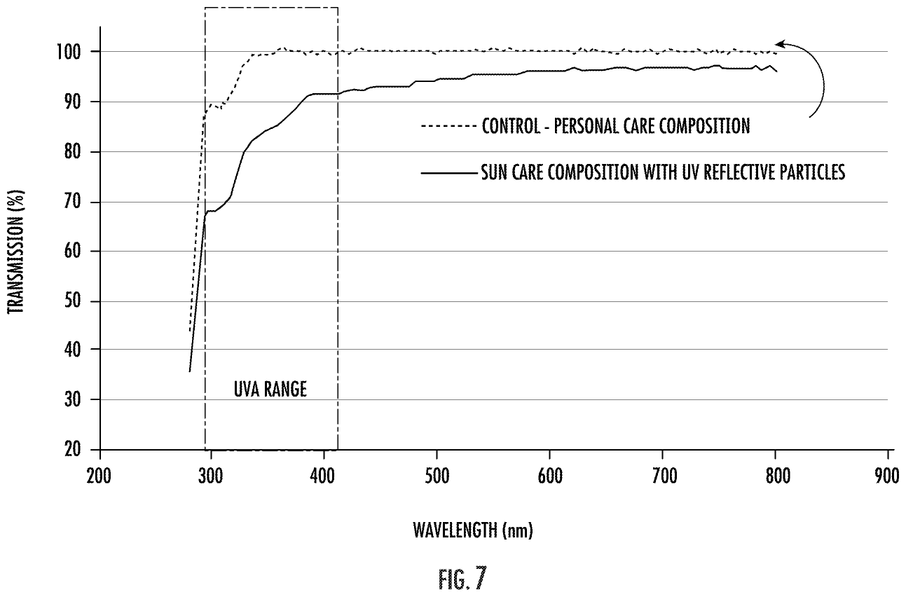

FIG. 7 is a UV-Vis transmission spectrum of the sun care composition and the control personal care composition.

As shown in FIG. 7, the sun care composition shows significantly lower transmission in the UV range (from 290 nm to 400 nm) as compared to the control sample. The sun care composition shows high transmission in the visible range (450 nm to 800 nm), which indicates desirable aesthetics on skin surface. Accordingly, a sun care composition containing UV reflective particles described herein would act to block harmful rays in the UV range.

It is understood that this disclosure is not limited to the embodiments specifically disclosed and exemplified herein. Various modifications of the invention will be apparent to those skilled in the art. Such changes and modifications may be made without departing from the scope of the appended claims. Moreover, each recited range includes all combinations and sub-combinations of ranges, as well as specific numerals contained therein.

1. A sun care composition, comprising:
UV reflective particles ground from an extruded film having a plurality of alternating layers of polycarbonate (PC) and poly(methyl methacrylate) (PMMA), wherein each layer is less than 150 nm thick; and
at least one of a cosmetically acceptable emollient, humectant, vitamin, moisturizer, conditioner, oil, silicone, suspending agent, surfactant, emulsifier, preservative, rheology modifier, pH adjustor, reducing agent, anti-oxidant, and/or foaming or de-foaming agent.

2. The sun care composition of claim 1, wherein the composition contains about 10% UV reflective particles by weight of the composition.

3. The sun care composition of claim 1, wherein the composition contains at least one of a humectant, a surfactant, and/or an emollient.

4. The sun care composition of claim 1, wherein the UV reflective particles block a portion of at least one of UVA radiation and/or UVB radiation.

5. The sun care composition of claim 1, wherein the sun care composition is highly transparent in the visible light range.

6. The sun care composition of claim 1, wherein the UV reflective particles have a median particle size less than 200 microns.

7. The sun care composition of claim 1, wherein the UV reflective particles have a median particle size of about 120 microns.

8. The sun care composition of claim 1, wherein the UV reflective particles have a median particle size of less than about 100 microns.

9. The sun care composition of claim 1, wherein the UV reflective particles have an aspect ratio of greater than about 1 to about 2.

10. The sun care composition of claim 1, wherein the UV reflective particles have an aspect ratio of about 1 to about 5.

11. The sun care composition of claim 1, wherein each of the individual polymer layers is less than 100 nm thick.

12. The sun care composition of claim 1, wherein each of the individual polymer layers has a thickness from about 50 nm to about 90 nm.

13. The sun care composition of claim 1, wherein the variation in thickness of each of the individual polymer layers is less than about 50%.

14. The sun care composition of claim 1, further comprising an additional sunscreen active.

15. The sun care composition of claim 1, further comprising one or more fragrance, propellant, opacifier/pearlizer, colorant, tanning agent, insect repellant, and/or biocide.

16. A method of protecting a skin of a mammal from damage caused by ultraviolet (UV) radiation, comprising:
applying, to the skin of the mammal, a sun care composition comprising:
UV reflective particles ground from an extruded film having a plurality of alternating layers of polycarbonate (PC) and poly(methyl methacrylate) (PMMA), wherein each layer is less than 150 nm thick; and
at least one of a cosmetically acceptable emollient, humectant, vitamin, moisturizer, conditioner, oil, silicone, suspending agent, surfactant, emulsifier, preservative, rheology modifier, pH adjustor, reducing agent, anti-oxidant, and/or foaming or de-foaming agent.

17. The method of claim 16, wherein the UV radiation is UVA radiation.

18. The method of claim 16, wherein the UV radiation is UVB radiation.

19. A sun care composition, comprising:
UV reflective particles ground from an extruded film having a plurality of alternating layers of polycarbonate (PC) and poly(methyl methacrylate) (PMMA), wherein each layer is less than 150 nm thick; and
at least one of a cosmetically acceptable emollient, humectant, or surfactant;
wherein the composition contains about 10% UV reflective particles by weight of the composition.

20. The sun care composition of claim 19, wherein the UV reflective particles have a median particle size less than 200 microns.
